(19) United States
(12) Patent Application Publication
(10) Pub. No.: US 2015/0006189 A1
(43) Pub. Date: Jan. 1, 2015

(54) Method and appartus for identifying possible treatment non-adherence

(71) Applicant: International Business Machines Corporation, Armonk, NY (US)

(72) Inventors: Liat Ein-Dor, Tel Aviv (IL); Jianying Hu, Bronx, NY (US); Martin Steven Kohn, East Hills, NY (US); Michal Ozery-Flato, Tel Aviv (IL); Michal Rosen-Zvi, Jerusalem (IL)

(73) Assignee: International Business Machines Corporation, Armonk, NY (US)

(21) Appl. No.: 13/932,022

(22) Filed: Jul. 1, 2013

(51) Int. Cl. G06F 19/00; A61B 5/00

(52) U.S. Cl. CPC G06F 19/3437; A61B 5/4833; A61B 5/746; G06F 19/345
USPC 705/2

(57) ABSTRACT

A computer-implemented method and apparatus for assessing treatment adherence by patients, the method comprising: receiving a model providing statistical significance of patients' response to treatment, the model based on treatment assigned to the patients, wherein the patients are diagnosed with a disease; computing by the computerized device a p-value for a result received for a patient diagnosed with the disease and being treated by the treatment, by applying the model to at least one patient; and issuing an alert responsive to the p-value being indicative of the result being unexpected beyond a threshold.

### TECHNICAL FIELD

The present disclosure relates to clinical decision support systems in general, and to a method and apparatus for identifying possible cases of non-adherence with treatment, in particular.

### BACKGROUND

A Clinical Decision Support System (CDSS or CDS) is a decision support system (DSS), which is designed to assist physicians and other health professionals with decision-making tasks, such as assigning tests or treatment for a patient. A clinical decision support system may be looked at as a knowledge system, which uses items of personal or medical data to provide medical case-specific advice.

An important factor in taking medical decision is the reaction of a patient to the prescribed treatment. Thus in order to determine the course of treatment for a patient, it is important to assess treatment adherence.

Treatment non-adherence is a growing concern to clinicians, healthcare systems, and other stakeholders (e.g., payers) because of mounting evidence that it is prevalent and associated with worsening of disease, death, and increased healthcare costs.

Treatment adherence may refer to whether patients adhere to their prescribed treatment. Treatment may refer to taking medications, keeping a prescribed diet, visiting a health professional such as a dietician, a physiotherapist or another health professional, or the like. For example, medication adherence may relate to taking the medications as prescribed in terms of frequency and dosage (e.g., one pill twice daily), as well as continuing to take the prescribed medication regularly. Adherence rates are typically lower among patients with chronic conditions, as compared with those with acute conditions.

Although medications are a specific case of treatment, the terms medication adherence (or non-adherence) and treatment adherence (or non-adherence) may be used interchangeably below and may relate to any treatment.

Currently there is no satisfying solution for recognizing medical non-adherence. Available methods for assessing adherence to medications can be generally divided into direct and indirect methods.

Direct methods include: directly observed therapy (e.g. observing patients taking pills or receiving records of the patient visiting a professional), measurement of concentrations of a drug or its metabolite in blood or urine, and detection or measurement in blood of a biologic marker added to the drug formulation. Although these direct methods are considered robust, they are problematic in routine clinical use.

Indirect methods of adherence assessment may include feedback from patients using for example patient questionnaires, self-reports, patient diaries or the like. However, self-report measures can be biased by inaccurate patient recall or by social desirability, whereby patients report an overly optimistic estimation of adherence to their healthcare providers.

Another indirect method relates to medication or treatment monitoring, for example pill counting, which does not accurately capture the exact timing of medication taking, electronic medication monitoring, which is not available to most patients and may not be an integral part of the data collected by healthcare systems, or assessing the rate of prescription refills, which data may not be available if the patient purchased the medication at a private pharmacy not by a pharmacy claim. These methods, although some of which may be easy to implement, do not provide a full solution. As for visiting health professionals out of the system, including doctor or other professional visits or out-of-system pharmacies, such records may not be available. In addition, data associated with some of the abovementioned methods can be manipulated by patients, for example by pill dumping, getting the medications but not taking them at all or not in accordance with the dosage or timing regime, etc.

Yet another group of methods relates to using the patient's clinical response as a measure for directly assessing the treatment adherence. However, the clinical response is known to be affected by factors other than adherence to a treatment regimen, resulting in such assessment being possibly not accurate.

In view of the above, there is a need in the art for a method and apparatus for assessing treatment adherence.

### BRIEF SUMMARY

A first aspect of the disclosed subject matter is a computer-implemented method performed by a computerized device, comprising: receiving a model providing statistical significance of patients' response to treatment, the model based on treatment assigned to the patients, wherein the patients are diagnosed with a disease; computing by the computerized device a p-value for a result received for a patient diagnosed with the disease and being treated by the treatment, by applying the model to at least one patient; and issuing an alert responsive to the p-value being indicative of the result being unexpected beyond a threshold.

Another aspect of the disclosed subject matter is an apparatus having a processing unit and a storage device, the apparatus comprising: a problem formulating component for formulating a problem related to a disease, a treatment for the disease and at least one response variable related to a patient state at an end of an evaluation period; a data receiving component for receiving a training set of a multiplicity of patients; a model assessment unit for assessing a model providing significance of patients' responses to the treatment, the model based on treatment assigned to the patients, wherein the patients are diagnosed with the disease; and a model application component for applying the model to at least one specific patient, for obtaining a p-value for a result received for the patient.

Yet another aspect of the disclosed subject matter is a computer program product comprising: a non-transitory computer readable medium; a first program instruction for receiving a model providing statistical significance of patients' response to treatment, the model based on treatment assigned to the patients, wherein the patients are diagnosed with a disease; a second program instruction for computing by the computerized device a p-value for a result received for a patient diagnosed with the disease and being treated by the treatment, by applying the model to at least one patient; and a third program instruction for issuing an alert responsive to the p-value being indicative of the result being unexpected beyond a threshold, wherein said first, second, and third program instructions are stored on said non-transitory computer readable medium.

### BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 shows a flow chart of steps in a method for identifying possible treatment non-adherence of a patient, in accordance with some exemplary embodiments of the disclosed subject matter; and

FIG. 2 shows a block diagram of components of an apparatus for identifying possible treatment non-adherence of a patient, in accordance with some exemplary embodiments of the disclosed subject matter.

### DETAILED DESCRIPTION

The disclosed subject matter is described below with reference to flowchart illustrations and/or block diagrams of methods, apparatus (systems) and computer program products according to embodiments of the subject matter. It will be understood that blocks of the flowchart illustrations and/or block diagrams, and combinations of blocks in the flowchart illustrations and/or block diagrams, can be implemented by computer program instructions. These computer program instructions may be provided to one or more processors of a general purpose computer, special purpose computer, a processor, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a non-transient computer-readable medium that can direct a computer or other programmable data processing apparatus to function in a particular manner, such that the instructions stored in the non-transient computer-readable medium produce an article of manufacture including instruction means which implement the function/act specified in the flowchart and/or block diagram block or blocks.

The computer program instructions may also be loaded onto a device. A computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

One technical problem dealt with by the disclosed subject matter is the need to assess the medication or treatment adherence of a patient, e.g., to what degree the patient takes the prescribed medications in accordance with the prescription, and keeps doing this durably. The terms medication and treatment may be used interchangeably in the description below, and may relate to any treatment or treatment combination.

Direct methods such as visual observations or substance concentration measurements in the patient's blood are impractical for prolonged periods of time. Indirect methods including patient querying, pill counting or electronic treatment monitoring also supply insufficient results.

Using the patient's clinical response as a measure is confounded by factors other than adherence to a treatment or medication regimen, and is thus possibly inaccurate. Therefore, without incorporating other information which includes confounding factors, the ability to identify possible non-adherence is limited.

In practice, physicians may suspect non-adherence if a patient's response is surprising or not as they expect. However, there is no systematic method for assessing adherence based on the extent to which the clinical result is expected or surprising.

One technical solution for detecting possible non-adherence comprises the assessment of the unexpectedness degree of a patient's clinical response. The input data specific for the patient may comprise the medications or treatment the patient is supposed to be taking and the appropriate regimen, and additional confounding factors that can be derived from the patient's medical records. Assessing the unexpectedness degree may use conditional anomaly detection methods, which may be based upon machine learning and statistical techniques.

The method and apparatus comprise a design and training phase. The training phase comprises formulation of the problem for a specific disease, and evaluating the treatment's effect on the patients' response.

When assessing the status of a specific patient, the measurements of the patient are assessed for unexpectedness, i.e., how surprising the results are relatively to the population of similar patients receiving similar treatment, or in other words, what is the p-value: the probability that observations as extreme as the given result (or worse) would occur by chance, under a given null hypothesis.

The assessment may be performed using unmediated methods, for example assessing the joint distribution of the relevant parameters. Alternatively, the assessment may use mediated methods, which may include constructing one or more prediction models, and then, given results for a specific patient, evaluating the significance of the prediction error.

The model may be further enhanced by assessing the effect of the treatment, and in particular drugs on the response under a given model. If changing the medication regimen in the model has no effect on the expected results, then the drugs and drug regimen are of no importance.

The model may be further enhanced with additional factors including non-adherence signals such as but not limited to lower frequency of visits to doctor's office or other non-urgent care facilities; larger variability in the time between visits to doctor's office or other non-urgent care facilities; lower frequency of pharmacy claims; larger variability in the time between pharmacy claims; larger variation in test results; depression as may be measured by diagnoses or medications; younger age; longer duration of the disease; or any other factor not used by the model, which factor may be associated with or indicative of non-adherence.

The model may then be applied to a specific patient, for assessing the p-value for the patient: how unexpected are the given results for the patient and the assigned treatment. If the results unexpected beyond a predetermined threshold, an alert may be fired.

One technical effect of the disclosed subject matter may relate to evaluating the treatment adherence of a patient, by assessing the p-value of the patient's results, or the probability of receiving such results or worse, which provides an indication to how surprising or unexpected the results are.

Low p-value, implying low probability for such results, may indicate an unexpected situation, such as a person not adhering with the prescribed treatment, such as medication regimen.

Another technical effect relates to combining the p-value with additional indications, some of which may relate to the patient's activity, such as doctor visits, pharmacy visits or others, which may increase or decrease the non-adherence suspicion.

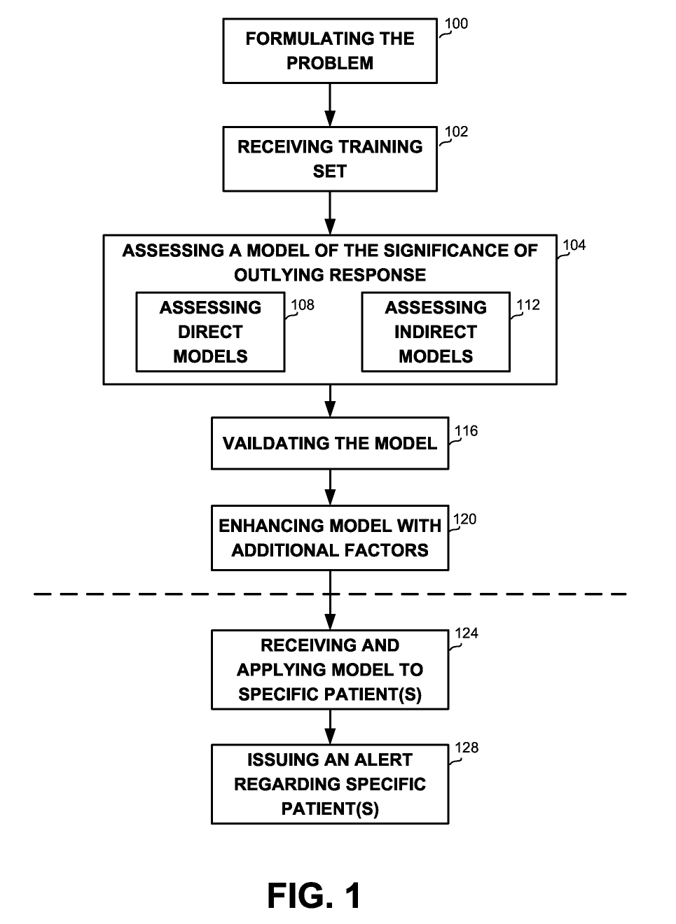

Referring now to FIG. 1, showing a flowchart of steps in a method for identifying possible medication non-adherence of a patient.

The first group of steps described below refers to a training or preparation stage, in which a model is prepared upon a training set comprising general information about the relevant disease as well as information about patients treated for the disease, including demographic details, treatment such as medication and regimen details, test results before or after the treatment, and possibly additional data.

On step 100, the problem at hand is formulated. Formulation may include but is not limited to any of the following:

The disease: the disease or medical condition, e.g., diabetes;

The treatment that may be prescribed for treating the disease or medical condition;

The test: a clinical or physical test which may be used to evaluate the effectiveness of one or more of the drugs, e.g., an HbA1c lab test for assessing the effectiveness of anti-diabetic drugs. Patients with chronic diseases often undergo periodical tests for assessing the degree of their illness and the effectiveness of the drugs prescribed to them;

Evaluation period: the period during which the patient is expected to adhere to the treatment regime. The defined test is performed at the start and end of the evaluation period, wherein the test at the beginning of the period may be referred to as the baseline test, and the test at the end of the period may be referred to as the final test. The period may be an approximately fixed duration, such as one month, three months, or the like;

The response: the response may be defined as the result of the final test or as a function of the baseline and final tests, e.g., the difference, ratio, or the like. The response may be assessed at the end of the evaluation period; and

Baseline information: any information derived from the patient's medical records of the patient prior to the evaluation period, such as demographic details such as gender, age or the like, medical information such as medical condition, prior treatment, additional diseases, or others and any other relevant information.

Formulation may also include defining any one or more of the following variables for each particular patient:

The response variable, which may be the same as the test result taken at the end of the evaluation period or any processing thereof;

The baseline test result variable;

The treatment or drugs variables as defined for the evaluation period, which may include mean dosage per drug, coverage per drug, and optionally additional variables that may account for possibly confounding factors, such as age, gender, clinical diagnoses, evaluation period length, mean prescribed dosage per drug in the evaluation or baseline period, mean dosage per drug collected from the pharmacy in the evaluation or baseline period, diagnoses in baseline period, test results in baseline period, age, gender, base result, and other drugs.

In some embodiments, variables containing information on clinical test results from the evaluation period may not be used, as they may provide information on the behavior of the patient during the evaluation period and thus interfere with the computation of the unexpectedness of the response.

The drugs variables and optionally additional variables which may be confounding, such as patient's age, gender, specific diagnoses or others may be referred to as predictor variables.

On step 102 a training set comprising patient data may be received from a storage device, a communication device, a communication channel, input by a user, or the like.

On step 104, the significance of the response to the drugs may be assessed. The significance may be a statistical score that corresponds to the observed response, and may be assessed on step 108 using unmediated models or on step 112 using mediated models, wherein each of the models provides the statistical significance of patients' response, given the assigned treatment and possibly one or more additional predictor variables.

Unmediated models may model the joint distribution of the variables including the response variable, and use it to evaluate the p-value of the response of a new patient, or in other words what is the probability to receive the test result of the patient or a worse result. Using formal notation, the p-value is P(Y≧y|X₁, . . . , Xₘ).

Mediated models learn a prediction model for the response variable, and may then use another model to evaluate the p-value for the prediction error.

Unmediated models, which may be constructed on step 108 may be implemented in a variety of ways, including but not limited to:

Gaussians mixture which models the response of a patient having given variables; or

K nearest neighbors which estimates a non-parametric distribution of the patient's response, using the responses of other patients and their corresponding distances from the particular patient in the variable space. It will be appreciated that the distances are based on the X vector and not on the response variable y.

Mediated models, which may be constructed on step 112 may also be implemented in a variety of ways, including but not limited to:

A prediction model: a prediction model may depend on the type of the response. For example, a classifier may be used for binary responses (an absent/present symptom), and a regression model may be used for a continuous response, or the like. It will be appreciated that a shorter evaluation period may generally imply expectation of the baseline test result variable to be more predictive.

Once the prediction model is determined, some alternatives exist for evaluating the significance of the prediction error, for example:

Estimating the p-value of the difference between the prediction and the actual result P(Yₜᵣᵤₑ−Y_{pred}≧d) (which is equivalent to P(Yₜᵣᵤₑ≧Y_{pred}+d=yₜᵣᵤₑ)) of the entire patient population, using standard parametric techniques, for example by assuming normal distribution of the difference Yₜᵣᵤₑ−Y_{pred};

Estimating the p-value using non-parametric techniques, for example by estimating the distribution of the difference Yₜᵣᵤₑ−Y_{pred} based on the training set; or

Estimating the conditional probability of the difference Yₜᵣᵤₑ−Y_{pred} given the predictor variables X, . . . Xₘ, i.e., P(Yₜᵣᵤₑ−Y_{pred}≧d|X, . . . Xₘ), using one of the unmediated methods described above, or any other unmediated method.

It will be appreciated that a model or a combination of two or more models may also be used, using one or more response variables. For example, K models (K>=2) may be used for estimating the joint probability P(Y₁ₜᵣᵤₑ≧y₁, . . . , Y_{Ktrue}≧y_{K}) or the conditional joint probability P(Y₁ₜᵣᵤₑ≧y₁, . . . , Y_{Ktrue}≧y_{K}|X₁, . . . Xₘ), where X, . . . Xₘ are the predictor variables; Y₁ₜᵣᵤₑ, . . . Yₖₜᵣᵤₑ are the response variables (which may be identical or different); and y₁, . . . , y_{K} are the observed response values for a patient. Each of the models may be mediated or unmediated. In some embodiments, a single model may be used, which uses two or more response variables.

It will be appreciated that a combination of two or more prediction models may also be used. For example, k prediction models (K>=2) may be used for estimating the joint probability P(Y₁ₜᵣᵤₑ−Y_{1pred}, . . . , Y_{Ktrue}−Y_{Kpred}) or the conditional joint probability P(Y₁ₜᵣᵤₑ−Y_{1pred}, . . . , Y_{Ktrue}−Y_{Kpred}|X₁, . . . Xₘ), where X₁, . . . Xₘ are the predictor variables.

Once the model, whether an unmediated or a mediated one is ready, it may be further validated on step 116.

One optional validation refers to validating that the treatment or drugs variables have effect on the response variable in a model that assesses the significance of an outlying response, i.e., verifying that the model indeed captures the effect of the drugs on the response.

Validation may be performed using any method, for example:

Non-parametric method, which may better suit mediated models or unmediated models that can operate as mediated models, e.g., are capable of providing predictions for the response given the predictor variables. The non-parametric method may comprise:

Defining a corresponding prediction model: for mediated methods such model may be the underlying prediction model. For unmediated methods, a prediction may be extracted from the joint distribution; and

Assessing the importance of the drugs variables by comparing the accuracy of the model with and without permuting the drugs variables. The accuracy of the models can be estimated by standard measurements such as area under the ROC curve (AUROC) for binary responses, mean square error (MSE) or coefficient of determination (R²) for continuous responses, or the like.

Another validation method, which may be used in parametric models comprises constructing two models, one with the drugs variables and the other without the drugs variables. The two models may be compared using statistical techniques for determining the best fitting model. The statistical techniques may include but are not limited to: likelihood ratio test, Akaike Information criterion or Bayesian Information criterion. Validation of the model would verify that the model with the drugs variables has a better fit.

On step 120, additional non-adherence factor may be incorporated for further assessing the non-adherence of patients. The indicators may include but are not limited to:

Lower frequency of doctor visits or other non-urgent care facilities;

Larger variability in the time between doctor visits or other non-urgent care facilities;

Lower frequency of pharmacy claims;

Larger variability in time between pharmacy claims;

Larger variation in lab test results or other test results;

Depression, which may be measured by y diagnoses or prescribed medications;

Younger age;

Longer duration of the disease; or

Any other factor not used by the model, which factor may be associated with or indicative of non-adherence.

The abovementioned parameters or signals and possibly additional ones may be too weak to be used for identifying individual non-adherent patients. However, when adjoined with the models above, and when considering large groups of patients it is more likely that a group of non-adherent patients would present stronger non-adherence signals than a group of adherent patients.

The abovementioned steps provide for constructing one or more models for assessing treatment non-adherence.

When a particular patient reaches the end of an evaluation term, the patient may take the tests, and the results may be evaluated.

Based upon the results, it may be required to assess whether the particular patient is adherent with the assigned regimen or not. In order to make the assessment, on step 124 the model constructed on steps 100, 108 or 112, and optionally 116 or 120 may be received and applied to the patient's results to obtain a p-value.

Alternatively, steps 100, 108 or 112, and optionally 116 or 120 may be performed separately or by another entity such as a research institute, and the model to be applied may be received rather than determined.

The p-value may be indicative of how surprising the patient's results are, e.g., to what degree they are different from what one would expect from similar patients receiving similar treatment.

On step 128 an alert or report may be issued regarding one or more patients having high probability to be medication non-adherent, indicating that the p-value or an associated measure is unexpected beyond a threshold. The report may be issued for each patient immediately after the model is applied to the patient's test results, after a predetermined number of tests for the same patient imply non-adherence, for a group of patients for example on a daily or weekly, basis or the like, or in any other arrangement.

Alerts can be made for all patients whose response p-value is below a certain threshold, wherein the threshold may be tuned for different levels of sensitivity. Alternatively, the threshold may be adjusted to the whole patient population, by using methods such as Bonferonni's correction for multiple hypotheses, or false discovery rate (FDR).

The alert or report may be exported to a database or a file, sent via e-mail to a caregiver or to the patient, or otherwise output by any method and to any destination.

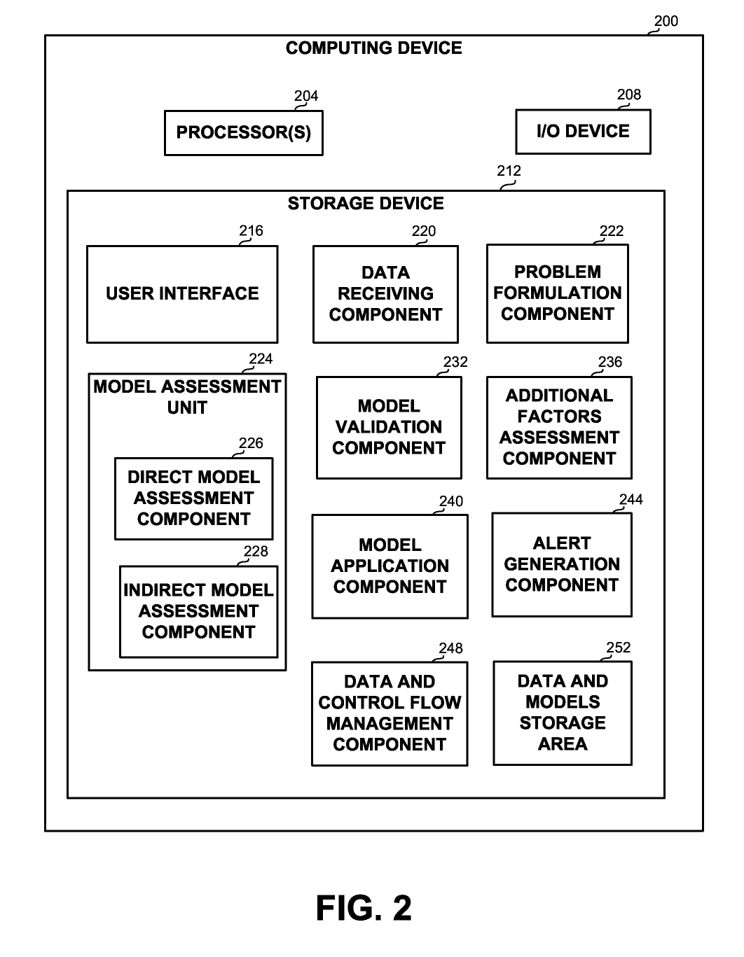

Referring now to FIG. 2 showing a block diagram of components of an apparatus for identifying possible medication non-adherence of a patient treated for a disease.

The environment comprises a computing device 200, associated with a health organization having a multiplicity of data records related to patients having a disease. Computing device 200 may comprise one or more processors 204. Any of processors 204 may be a Central Processing Unit (CPU), a microprocessor, an electronic circuit, an Integrated Circuit (IC) or the like. Alternatively, computing device 200 can be implemented as firmware written for or ported to a specific processor such as digital signal processor (DSP) or microcontrollers, or can be implemented as hardware or configurable hardware such as field programmable gate array (FPGA) or application specific integrated circuit (ASIC). Processors 204 may be utilized to perform computations required by comptuing device 200 or any of it subcomponents.

In some embodiments, computing device 200 may comprise an input-output (I/O) device 208 such as a terminal, a display, a keyboard, a touch screen, an input device or the like to interact with the system, to invoke the system and to receive results. It will however be appreciated that the system can operate without human operation and without I/O device 208.

Comptuing device 200 may comprise one or more storage devices 212 for storing executable components, and which may also contain data during execution of one or more components. Storage device 212 may be persistent or volatile. For example, storage device 212 can be a Flash disk, a Random Access Memory (RAM), a memory chip, an optical storage device such as a CD, a DVD, or a laser disk; a magnetic storage device such as a tape, a hard disk, storage area network (SAN), a network attached storage (NAS), or others; a semiconductor storage device such as Flash device, memory stick, or the like. In some exemplary embodiments, storage device 212 may retain program code operative to cause any of processors 204 to perform acts associated with any of the steps shown in FIG. 1 above, for example building a model.

Storage device 212 may comprise or be in communication with one or more additional storage areas for storing patient data, test results or other data associated with the apparatus.

The components detailed below may be implemented as one or more sets of interrelated computer instructions, loaded to storage device 212 and executed for example by any of processors 204 or by another processor. The components may be arranged as one or more executable files, dynamic libraries, static libraries, methods, functions, services, or the like, programmed in any programming language and under any computing environment.

Storage device 212 may comprise or be loaded with user interface 216 adapted to let a user of the system enter data and receive results, optionally through usage of and of I/O device 208. User interface 216 may be adapted to display options or data to a user, receive user selections or data, and provide the data to other components such as storage, calculation units or the like.

Storage device 212 may also comprise or be loaded with data receiving component 220 for receiving data such as patient data, disease or medication data or the like. The data may be received from a storage device, from another system or form any other source and in any format. The received data may include a training set upon which one or more models may be constructed, and data related to a patient to be evaluated.

In some alternative embodiments, if the model is determined at another time or location, Storage device 212 may comprise a the model may be received instead of the data

Storage device 212 may also comprise or be loaded with problem formulation component 222 adapted for formulating the underlying problem as described in association with step 100 of FIG. 1. Formulation may use data received from a user identifying the disease, and the medication, defining the evaluation time, and the like.

Storage device 212 may also comprise model assessment unit 224, which calculates one or more models based on the training set received by data receiving component 220. Model assessment unit 224 may comprise unmediated model assessment component 226 for assessing or determining a unmediated model as described in association with step 108 of FIG. 1, or mediated model assessment component 228 for assessing or determining an mediated model as described in association with step 112 of FIG. 1

It will be appreciated that model assessment unit 224 may comprise either one or both of unmediated model assessment component 226 and mediated model assessment component 228, and the component or components to be used may be determined in accordance with the model to be assessed, which may depend on available data, the disease type, or the like.

Storage device 212 may also comprise or be loaded with model validation component 232 for validating the determined or assessed model or models. Validation may be used for a variety of aspects, such as validating that the drugs variables have effect on the response variable in the constructed model, as described in association with step 116 of FIG. 1.

Storage device 212 may comprise additional factors assessment component 236 for assessing additional factors which may be associated with the patients' adherence or non-adherence to medication regimen, as described in association with step 120 of FIG. 1

Storage device 212 may also comprise model application component 240 for applying the determined one or more models towards assessing for a particular patient or patients whether they may be medication non-adherent. The non-adherence may be determined according to the p-value of the results, e.g., the probability of similar patients to exhibit such test results or worse. This assessment may further be crossed with additional factors such as doctor or pharmacy visits, age and other factors discussed above.

Storage device 212 may further comprise alert generation component 244 for generating an alert or a report regarding one or more patients that have been indicated as possibly non-adherent. A report may be sent anytime a patient is identified as possibly non-adherent, or every day, week, month or the like and detailing all patients identified in this period, or the like.

Storage device 212 may also comprise data and control flow management component 248 for activating components, managing the data flow between the components, synchronizing and timing activities, or the like.

Storage device 212 may also comprise or be in communication with data and models storage area 252 storing the raw data, patient data, the assessed models, results of applying models to patients, or the like.

It will also be appreciated that the disclosed method and apparatus may be used for other purposes such as identifying patients with unexpectedly good results which may be interesting to further explore, or others.

The flowchart and block diagrams in the figures illustrate the architecture, functionality, and operation of possible implementations of systems, methods and computer program products according to various embodiments of the present disclosure. In this regard, each block in the flowchart and some of the blocks in the block diagrams may represent a module, segment, or portion of program code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As will be appreciated by one skilled in the art, the disclosed subject matter may be embodied as a system, method or computer program product. Accordingly, the disclosed subject matter may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product embodied in any tangible medium of expression having computer-usable program code embodied in the medium.

Any combination of one or more computer usable or computer readable medium(s) may be utilized. The computer-usable or computer-readable medium may be, for example but not limited to, any non-transitory computer-readable medium, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CDROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, and the like.

Computer program code for carrying out operations of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A computer-implemented method performed by a computerized device, comprising:
receiving a model providing statistical significance of patients' response to treatment, the model based on treatment assigned to the patients, wherein the patients are diagnosed with a disease;
computing by the computerized device a p-value for a result received for a patient diagnosed with the disease and being treated by the treatment, by applying the model to at least one patient; and
issuing an alert responsive to the p-value being indicative of the result being unexpected beyond a threshold.

2. The computer-implemented method of claim 1, wherein the model is also based on at least one predictor variable.

3. The computer-implemented method of claim 2, wherein the at least one predictor variable is selected from the group consisting of: evaluation period length, mean prescribed dosage per drug in the evaluation or baseline period, mean dosage per drug collected from the pharmacy in the evaluation or baseline period, diagnoses in baseline period, test results in baseline period, age, gender, and base result.

4. The computer-implemented method of claim 1 further comprising:
formulating a problem related to the disease, treatments for the disease and at least one response variable related to a patient state at an end of an evaluation period;
receiving a training set of a multiplicity of patients; and
assessing the model of the significance of patients' responses.

5. The computer-implemented method of claim 4, wherein assessing the model of the response significance comprises assessing at least one unmediated model or at least one mediated model.

6. The computer-implemented method of claim 4, further comprising validating the model.

7. The computer-implemented method of claim 6, wherein validating the model comprises validating that the model captures an effect of the treatment on the at least one response variable.

8. The computer-implemented method of claim 5, further comprising enhancing the model with at least one factor.

9. The computer-implemented method of claim 7, wherein the at least one factor is selected from the group consisting of: low frequency of doctor visits or other non-urgent care facilities visits; larger variability in time between doctor visits or other non-urgent care facilities; lower frequency of pharmacy claims; larger variability in time between pharmacy claims; larger variation in test results; depression; young age; and long duration of the disease.

10. The computer-implemented method of claim 1 wherein the treatment is a prescribed medication.

11. The computer-implemented method of claim 1 wherein the treatment is a prescribed diet or a reoccurring visit to a healthcare professional.

12. An apparatus having a processing unit and a storage device, the apparatus comprising:
a problem formulating component for formulating a problem related to a disease, a treatment for the disease and at least one response variable related to a patient state at an end of an evaluation period;
a data receiving component for receiving a training set of a multiplicity of patients;
a model assessment unit for assessing a model providing significance of patients' responses to the treatment, the model based on treatment assigned to the patients, wherein the patients are diagnosed with the disease; and
a model application component for applying the model to at least one specific patient, for obtaining a p-value for a result received for the patient.

13. The apparatus of claim 12, wherein the model is also based on at least one predictor variable.

14. The apparatus of claim 13, wherein the at least one predictor variable is selected from the group consisting of: evaluation period length, mean prescribed dosage per drug in the evaluation or baseline period, mean dosage per drug collected from the pharmacy in the evaluation or baseline period, diagnoses in baseline period, test results in baseline period, age, gender, and base result.

15. The apparatus of claim 12, further comprising an alert generation component for issuing an alert responsive to the p-value being indicative of the result being unexpected beyond a threshold.

16. The apparatus of claim 12, wherein the model assessment unit comprises an unmediated model assessment unit for assessing an unmediated model of the response significance or a mediated model assessment unit for assessing a mediated model of the response significance.

17. The apparatus of claim 12, further comprising a model validation component for validating the model.

18. The apparatus of claim 17, wherein validating the model validation component is adapted to validate that the model captures an effect of the treatment on the at least one response variable.

19. The apparatus of claim 12, further comprising a factor assessment component for enhancing the model with at least one factor.

20. A computer program product comprising: a non-transitory computer readable medium;
a first program instruction for receiving a model providing statistical significance of patients' response to treatment, the model based on treatment assigned to the patients, wherein the patients are diagnosed with a disease;
a second program instruction for computing by the computerized device a p-value for a result received for a patient diagnosed with the disease and being treated by the treatment, by applying the model to at least one patient; and
a third program instruction for issuing an alert responsive to the p-value being indicative of the result being unexpected beyond a threshold,
wherein said first, second, and third program instructions are stored on said non-transitory computer readable medium.
